(19) United States
(12) Patent Application Publication
(10) Pub. No.: US 2015/0231130 A1
(43) Pub. Date: Aug. 20, 2015

(54) PHARMACEUTICAL COMPOSITION COMPRISING DIAMORPHINE FOR INTRANASAL ADMINISTRATION

(71) Applicants: Neil Wynne, Wrexham (GB); Sirjiwan Singh, Wrexham (GB)

(72) Inventors: Neil Wynne, Wrexham (GB); Sirjiwan Singh, Wrexham (GB)

(73) Assignee: WOCKHARDT LIMITED, Aurangabad 0, (IN)

(21) Appl. No.: 14/419,524

(22) PCT Filed: Oct. 8, 2012
(86) PCT No.: PCT/IB2012/055421

§ 371 Date: Feb. 4, 2015

(30) Foreign Application Priority Data
Jul. 26, 2012 (IN) 2147/MUM/2012

(51) Int. Cl. A61K 31/485; A61M 15/08; A61M 11/00; A61K 9/00; A61M 15/00

(52) U.S. Cl. CPC A61K 31/485; A61K 9/0043; A61M 15/009; A61M 11/007; A61M 15/08; A61M 2202/0468; A61M 2210/0618; A61M 2207/00

(57) ABSTRACT

The invention provides a method of assembling a device and a kit suitable for administration to multiple patients comprising a multi-dose pharmaceutical composition of nasal analgesic in the form of a nasal spray. The present invention also provides a method for reducing the respiratory depressant effect associated with diamorphine administration, the method comprise a single dose pharmaceutical composition comprising lyophilized diamorphine and a diluent for reconstitution, wherein the dose of diamorphine is 0.1 mg/kg body weight.

### FIELD OF THE INVENTION

The invention provides a method of assembling a device and a kit suitable for administration to multiple patients without cross-contamination, comprising a multi-dose pharmaceutical composition comprising diamorphine or a pharmaceutically acceptable salt thereof, wherein the composition is adapted for intranasal delivery using a nasal spray. The present invention also provides a method for reducing the respiratory depressant effect associated with diamorphine administration, the method comprise a single dose pharmaceutical composition comprising lyophilized diamorphine and a diluent for reconstitution, wherein the dose of diamorphine is 0.1 mg/kg body weight.

The invention also provides a method for treating pain by administering intranasally a pharmaceutical compositions comprising diamorphine or a pharmaceutically acceptable salt thereof wherein the said composition is adapted for delivery of a single dose of diamorphine in an amount of about 0.1 mg/kg diamorphine or a pharmaceutically acceptable salt thereof per kg body weight of a patient.

### BACKGROUND OF THE INVENTION

Pain is an unpleasant sensation often caused by intense or damaging stimuli such mechanical, thermal, chemical or other noxious stimuli. Pain is experienced when the free nerve endings, which constitute the pain receptors in the skin as well as in certain internal tissues. The pain receptors (nociceptors) can transmit signals along afferent neurons into the central nervous system and then to the brain.

Pain is the most common symptom for which patients seek medical advice and treatment. Pain can be acute or chronic. While acute pain is usually self-limited, chronic pain can persist longer and lead to significant changes in a patient's personality, lifestyle, functional ability or overall quality of life.

Most pain resolves promptly once the painful stimulus is removed and the body has healed, but sometimes pain persists despite removal of the stimulus and apparent healing of the body and sometimes pain arises in the absence of any detectable stimulus, damage or disease.

A wide variety of compounds can act as analgesics. Two important classes of analgesics are opioid analgesics and non-steroidal anti-inflammatory drugs (NSAIDs).

An opioid is a psychoactive chemical that works by binding to opioid receptors, which are found principally in the central and peripheral nervous system and the gastrointestinal tract. The receptors in these organ systems mediate both the beneficial effects and the side effects of opioids.

Opioids are among the worlds oldest known drugs the use of the opium poppy for its therapeutic benefits predates recorded history. The analgesic (painkiller) effects of opioids are due to decreased perception of pain, decreased reaction to pain as well as increased pain tolerance.

Opioid analgesics exhibit morphine-like properties and can be sub-classified on the basis of their receptor specificity. They act as an agonist primarily at mu, kappa and perhaps delta receptors in the central nervous system. By acting on these receptors, they cause analgesia and anesthesia as a result of a receptor-mediated central action on pain perception, together with a receptor-medicated modulatory effect on the central transmission of noxious sensation.

Diacetylmorphine, also known as diamorphine (BAN), is an opioid analgesic synthesized from morphine, a derivative of the opium poppy. When used in medicine it is typically used to treat severe pain, such as that resulting from a heart attack. It is the 3,6-diacetyl ester of morphine, and functions as a morphine prodrug (meaning that it is metabolically converted to morphine inside the body). The white crystalline form considered “pure diamorphine” is usually the hydrochloride salt, diacetylmorphine hydrochloride.

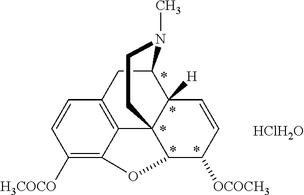

It is a potent opiate analgesic, which has a more rapid onset of activity than morphine as the first metabolite, monoacetylmorphine, more readily crosses the blood brain barrier. In man, diamorphine has a half-life of two to three minutes. Its first metabolite, monoacetylmorphine, is more slowly hydrolyzed in the blood to be concentrated mainly in skeletal muscle, kidney, lung, liver and spleen. Monoacetylmorphine is metabolized to morphine. Diamorphine does not bind to protein. However, morphine is about 35% bound to human plasma proteins, mainly to albumin. The analgesic effect lasts approximately three to four hours.

Diamorphine as a narcotic analgesic acts primarily on central nervous system and smooth muscles. Diamorphine is available in form of supplied in tablet and in 5 mg, 30 mg, 100 mg and 500 mg ampoules of freeze dried diamorphine. It is know in the art that diamorphine is highly unstable in liquid dosage form. In presence of water, diamorphine hydrochloride degrades. After hydrolysis, the initial degradation products are 6-O-acetylmorphine and acetic acid. The 6-acetyl group hydrolyses slowly to morphine. The commercially known Injectable products of diamorphine recommend immediate use of the product after reconstitution. The labels clearly mention the storage of reconstituted product should normally not be longer than 24 hours at 2-8° C.

As with other opioids, diacetylmorphine is used as both an analgesic and a recreational drug. Diamorphine Lyophillisate for solution for injection is indicated in the treatment of severe pain associated with surgical procedures, myocardial infarction or pain in the terminally ill and for the relief of dyspnoea in acute pulmonary oedema. Frequent and regular administration is associated with tolerance and physical dependence, which may develop into addiction. Internationally, diacetylmorphine is controlled under Schedules I and IV of the Single Convention on Narcotic Drugs.

Injectable dosage form suffers from various disadvantages such as pain, bruises, allergic reactions, self-administration not possible, etc. Moreover, intravenous injection is generally associated with rapid offset of pain relief as the circulating analgesic is cleared from the plasma.

Different routes of administration other than solid and Injectable dosage form include nasal, transdermal, transocular administration, transrectal administration, transpulmonary administration, etc. Among these non-injection type administration methods, nasal administration offer several advantages such as that it is easy, well-tolerated, non-invasive transmucosal route which avoids first pass metabolism of the drug in liver and provides rapid relief. But the drug loses associated with nasal administration are more compared to intravenous administration. Injectable and nasal routes are recognized routes for administration of diamorphine. The usual dose of diamorphine through injectable and nasal routes is 0.1 mg/kg to about 0.2 mg/kg body weight.

Use of 0.1 mg/kg to about 0.2 mg/kg body weight doses of diamorphine for producing analgesia is limited because of the side effects associated with it. One of the major life-threatening side effect associated with morphine or diamorphine administration through recognized routes of administration is respiratory depression. Diamorphine is the 3,6-diacetyl ester of morphine. Diamorphine in-vivo converts to morphine. Diamorphine or morphine when administered, has the potential to slow the rate of breathing and, ultimately, to stop breathing altogether. This is the main concern for a doctor in deciding whether to or not to prescribe diamorphine, if so, how much to prescribe. If a patient already has a condition causing respiratory depression, such as chest disease, the further depressant effect of the drug on the patient's breathing may easily give rise to danger. The respiratory depressant effect is reported with even at lower doses such as 0.1 mg/kg of body weight of diamorphine after intravenous injection. Thus, there is a need of a method for reducing the respiratory depressant effect associated with diamorphine administration.

PCT Patent Application No. 2009040595A1 discloses a multi-dose pharmaceutical composition for intranasal administration in the form of a nasal spray comprising lyophilized nasal analgesic and suitable diluent for reconstitution.

PCT Patent Application No. 2005004961A1 discloses a dispenser comprising a reservoir containing a plurality of dosage units each of which comprise a formulation of a controlled drug or a drug of abuse, said dosage units being contained in a tamper-evident manner such that access to the dosage units in use is controlled either by the dispenser or remotely and/or is monitored either by the dispenser or remotely.

U.S. Pat. No. 5,843,480 disclose a controlled-release pharmaceutical preparation comprising diamorphine, or a pharmaceutically acceptable salt thereof.

PCT Patent Application No. 9802148 discloses a complex of diamorphine-polyacrylate and cocaine-polyacrylate.

PCT Patent Application No. 2008092267 discloses a composition comprising an opioid receptor agonist in an amount effective to produce a therapeutic effect and a cannabinoid receptor antagonist in an amount effective to potentiate a therapeutic activity of an opioid receptor agonist and/or inhibit, delay, reduce and/or reverse tolerance to the opioid receptor agonist.

US Patent Application No. 20050053647 discloses a pharmaceutical device for continuous and controlled release of at least one active substance for application to the undamaged skin, to the oral, lingual, nasal or rectal mucosae, to the bronchial or alveolar epithelium, or parenterally with inclusion of an absorption process, wherein the active substance is diamorphine, which is present as diamorphine base, in the form of a pharmaceutically compatible acid addition salt or in the form of an inclusion compound.

EP Patent No. 1722759B1 discloses a nasal or ocular drug delivery composition in the form of an aqueous solution or suspension for delivery of a therapeutic agent across a nasal or ocular mucosal surface into the systemic circulation comprising chitosan, a salt thereof or a derivative thereof that has been formed by bonding of acyl or alkyl groups with the hydroxyl groups of the chitosan or a salt of such a derivative thereof; a polyol-phosphate or sugar-phosphate salt; triethyl citrate as a plasticizer; and a systemically acting therapeutic agent.

Adam et al., Oxford handbook of Critical Care Nursing, Chapter 3, pg 50-51, discloses use of opoid analgesics for the management of pain with diamorphine dose of 0.05-0.1 mg/kg by intravenous route.

Alexander-Williams J. M. et al. (Br. J. Anaesth. 1998; 81: 3-7) disclose novel routes of opioid administration.

Mitchell et al. (Eur Addict Res 2006; 12: 91-95) discloses feasibility and acceptability of an intranasal diamorphine spray as an alternative to injectable diamorphine.

Wilson et al. (J Accid Emerg Med 1997; 14: 70-72) discloses the safety and efficacy of 0.1 mg/kg of intranasal diamorphine as an analgesic for use in children in accident and emergency.

Hallett et al. (Anaesthesia, 2000, 55, 532-539), discloses use of intranasal diamorphine for postoperative pain as an adjunct to the other drugs.

Kendall et al. (British Medical Journal, 322: 261-265) compares the effectiveness and safety profile of a dose of 0.1 mg/kg of diamorphine nasal spray with 0.2 mg/kg of intramuscular morphine for managing acute pain in children and teenagers with a clinical fracture.

Wyatt et al. (Oxford handbook of Emergency Medicine, 3^{rd} Ed., pg 279) discloses a dose of 0.1 mg/kg of nasal diamorphine for providing analgesia in children.

Telfer et al. (Arch Dis Child 2009; 94: 979-980) discloses use of intranasal diamorphine when given in combination with intravenous or oral morphine for rapid analgesia.

Ward et al. (Anaesthesia 2002; 57 (1): 49-52) discloses the effectiveness of diamorphine administered either intravenously (0.5 mg bolus) or intranasally (1.0 mg bolus) for patient controlled analgesia during the early postoperative period.

Kerr, Maya; Maconochie, Ian (Intranasal diamorphine usage in Paediatric accident and emergency. Current Pediatric Reviews (2010), 6(3), 151-155) discloses intranasal diamorphine is widely accepted and the most commonly used analgesia in Paediatric A&E for acute pain in long bone fracture.

Kendall, Jason M.; Latter, Victoria S. (Intranasal diamorphine as an alternative to intramuscular morphine: pharmacokinetic and pharmacodynamic aspects. Clinical Pharmacokinetics (2003), 42(6), 501-513) discloses pharmacokinetic profile of intranasal diamorphine in adults.

Regan Luke et al. (Nose and vein, speed and pain: comparing the use of intranasal diamorphine and intravenous morphine in a Scottish paediatric emergency department. Emergency medicine journal: EMJ (2012)) compared the clinical performance of Intranasal Diamorphine with Intravenous morphine.

PCT Patent Application Nos. 2000076477 and 2006016530, U.S. Pat. No. 4,464,378, and U.S. Pat. No. 6,677,346, US Patent Application No. 2006110333, disclose morphine and morphine derivatives in the form of lyophilized powder for nasal administration with the aid of nasal insufflator or jet-spray.

U.S. Pat. No. 6,608,073 discloses codeine optionally in combination with opioid analgesics in the form of solution or gel for nasal administration with the aid of finger or cotton tipped applicator.

U.S. Pat. No. 5,756,483 discloses apomorphine and morphine solution or gel for nasal administration with the aid of nasal tampon or nasal sponge.

U.S. Pat. No. 4,973,596 discloses meperidine solution with a single dose dispenser.

U.S. Pat. No. 4,703,864 provides for a unitary molded plastic cover for a container such as medicament bottle, in which removable cap portion is attached by a severable tear strip.

U.S. Pat. No. 5,350,116 provides for an actuator for a liquid spray pump provided with a skirt, which cooperates with the body of the pump to compress a volume of air during pump actuation.

U.S. Pat. No. 5,509,578 provides for pump, which has a tubular portion which is arranged to penetrate the mouth of a container having a seal in order to dispense liquid from the container.

U.S. Pat. No. 6,948,492 and US Patent Application No. 2006/0021614 provide for an apparatus and method for the self-administration of a plurality of doses of an intranasal liquid pharmaceutical composition including opioid analgesics that includes a drug delivery device containing a plurality of sealed vials.

PCT Patent Application No. 2009040595A1 discloses a multi-dose pharmaceutical composition for intranasal administration in the form of a nasal spray comprising lyophilized nasal analgesic and suitable diluent for reconstitution.

PCT Patent Application No. 2008152398A1 discloses the aerosol formulation comprising an active material coated with a polymer, in combination with a propellant, optionally with other pharmaceutically acceptable excipients. Formulation given in example

US Patent Application No. 20080248991A1 discloses a composition having a viscosity of 150 cp or less at 25° C. and comprising (i) chitosan, a salt or derivative thereof or a salt of a derivative thereof, (ii) a polyol-phosphate or sugar-phosphate salt, (iii) a plasticizer, and (iv) a therapeutic agent. Examples discloses the compositions.

The present invention attempts to overcome the problems associated with Diamorphine administration by providing a multi-dose pharmaceutical composition, which reduces the anti-depressant effect of diacerein. The invention also provides a kit comprising multi-dose pharmaceutical composition and replaceable nasal tips so that the multi-dose pharmaceutical composition can be given to multiple patients without cross-contamination by replacing the nasal tip after each administration.

### SUMMARY OF THE INVENTION

One of the embodiments of the present invention provides method of reducing the respiratory depressant effect of diamorphine wherein the method comprises administering intranasally a single dose pharmaceutical composition comprising (a) lyophilized diamorphine or pharmaceutically acceptable salts thereof, and (b) a diluent for reconstitution; wherein the diluent for reconstitution comprises a preservative, a chelating agent, one or more osmolarity modifiers to maintain the osmolarlity of the said composition between 250-500 Osm/L and pH modifiers to maintain the pH of the said composition between 3.5 to 6.5, wherein the dose of diamorphine is 0.1 mg/kg body weight.

The diluent for reconstitution for the purpose of present invention is water or saline comprising comprises a preservative, a chelating agent, one or more osmolarity modifiers or pH modifiers.

Another embodiment of the present invention provides a method for treating pain, said method comprising administering intranasally a pharmaceutical composition comprising diamorphine or a pharmaceutically acceptable salt thereof, reconstituted with a diluent for reconstitution, said composition adapted for a delivery of a single dose of diamorphine or a pharmaceutically acceptable salt thereof in an amount of 0.1 mg diamorphine or a pharmaceutically acceptable salt thereof per kg body weight of a patient.

Another embodiment of the present invention provides a pharmaceutical composition comprising diamorphine or a pharmaceutically acceptable salt thereof, wherein the composition is adapted for intranasal delivery of diamorphine or a pharmaceutically acceptable salt thereof using a nasal spray capable of delivering diamorphine or a pharmaceutically acceptable salt thereof in an amount of 0.1 mg/kg body weight.

Another embodiment of the present invention provides a method of assembling a device suitable for administration to multiple patients a multi-dose pharmaceutical composition of nasal analgesic in the form of a nasal spray, wherein the method comprises: (a) attaching the modified nasal pump to the bottle by hand so that it fits tightly into a bottle to prevent leakage; (b) shaking the assembly of step (a) for reconstitution of the lyophilized nasal analgesic; (c) priming the assembly of step (b) to release the intended quantity of the reconstituted nasal analgesic; (d) inserting the nasal tip into the patient no. 1's nostril and directing the spray to the sidewall of the nose of patient; (e) replacing the used nasal tip of step (d) with new nasal tip; (f) priming the assembly to release the intended quantity of the reconstituted nasal analgesic, and (g) inserting the nasal tip into the patient no. 2's nostril and directing the spray to the sidewall of the nose of patient.

Another embodiment of the present invention provides a method for reducing the cross-contamination while using a multi-dose pharmaceutical composition of nasal analgesic in the form of a nasal spray in multiple patients by providing a kit comprising multi-dose pharmaceutical composition and replaceable nasal tips; wherein the method comprises (a) inserting the nasal tip of the nasal spray comprising the pharmaceutical composition into the patient no. 1's nostril and directing the spray to the sidewall of the nose of patient; (b) replacing the used nasal tip of step (a) with new nasal tip; (c) inserting the nasal tip into the patient no. 2's nostril and directing the spray to the sidewall of the nose of patient and (d) repeating the step (a)-(c) for each new patient.

In another embodiment of the present invention there is provided a kit for the preparation of multi-dose pharmaceutical composition of diamorphine of the present invention in the form of a nasal spray comprising a bottle containing lyophilized nasal analgesic, a tube containing diluent for reconstitution, modified nasal pump, nasal tips and pack insert or a label, which provides directions for assembling the kit and the subsequent use.

### DESCRIPTION OF THE DRAWINGS

FIG. 1 shows exploded view of the kit package with the components and their use.

FIG. 2 shows a typical modified nasal pump.

FIG. 3 shows a typical bottle to be attached to the nasal pump.

### DETAILED DESCRIPTION OF THE INVENTION

In an attempt to develop a safe and effective multi-use intranasal diamorphine composition, applicants have surprisingly found that when a single dose pharmaceutical composition comprising lyophilized diamorphine reconstituted with a diluent for reconstitution, wherein the dose of diamorphine is about 0.1 mg/kg body weight was administered to a patient in need of a pain relief, the adverse effects associated with diamorphine administration were found to be very low. No respiratory depressant effect was observed in patients during the study by the administration of the single dose of diamorphine is 0.1 mg/kg body weight. At the same time this dose of 0.1 mg/kg body weight of diamorphine intranasally as compared to standard diamorphine intravenous formulation was found to be effective alone in producing the analgesic effects in children aged from 1-<16 years attending hospital for minor surgery, laser treatment and requiring intravenous cannulation and opiate analgesia during routine clinical management.

As the 0.1 mg/kg body weight dose of diamorphine when administered in form of a pharmaceutical composition is free of major side effects, it can be used for producing the patient controlled analgesia in post-operative conditions without the assistance of doctor and ambulatory services.

Moreover, the pharmaceutical composition of the present invention were found to be stable for atleast three weeks after reconstitution and does not require stringent storage conditions. The compositions of the present invention are suitable for multiple-use in the form of nasal spray for the treatment of pain in an emergency setting. The compositions of the present invention are found to be safe and efficacious in-vivo. The nasal administration provides fast onset of analgesia similar to injection, increases the time of residence of diamorphine in nasal cavity, thus resulting in prolonged analgesia.

When multi-dose nasal compositions are administered to number of patient, it leads to cross contamination among patients typically in a hospital setting, which can lead to serious mishaps. To avoid the same, the present inventors have also developed a device suitable for administration to multiple patients a multi-dose pharmaceutical composition of nasal analgesic in the form of a nasal spray without cross-contamination and can be utilized easily for use in different patients. The assembly not only avoids cross-contamination but also maintains the integrity of the formulation throughout use.

The pharmaceutical compositions of the present invention are adapted for use in pre-hospital ambulatory conditions by paramedics. Pre-hospital ambulatory conditions such as clinical fractures occurring during mountaineering, racing, driving, etc. which requires instant pain relief can suitably be treated by administering intranasally pharmaceutical composition comprising diamorphine. The use of compositions of present invention does not require any hospitalization. Further, paramedics need no special training for administering the diamorphine compositions intranasally. The pharmaceutical compositions of the present invention are useful in postoperative pain.

The present invention provides method of reducing the respiratory depressant effect of diamorphine wherein the method comprises administering intranasally a single dose pharmaceutical composition comprising (a) lyophilized diamorphine hydrochloride, and (b) a diluent for reconstitution; wherein the diluent for reconstitution comprises a preservative, a chelating agent, one or more osmolarity modifiers to maintain the osmolarlity of the said composition between 250-500 Osm/L and pH modifiers to maintain the pH of the said composition between 3.5 to 6.5, wherein the dose of diamorphine is 0.1 mg/kg body weight.

The pharmaceutical composition of the present invention is a liquid dosage form.

The pharmaceutical composition of the present invention is reconstituted liquid dosage form.

The dose of diacerein for the purpose of present invention varies from about 0.05 mg/kg to 0.2 mg/kg body weight.

Suitable preservatives include but are not limited to m-cresol, phenol, alcohol, benzyl alcohol, methyl-, ethyl-, propyl- and butyl-paraben, thiomersal, chlorobutanol, benzalkonium or any combination thereof. Typically, the preservative may be present in the formulations in a concentration of from about 0.001% up to about 5% by weight of the total formulation.

The preservatives or chelating agents added to the diluent for reconstitution do not avoid the degradation of diamorphine in solution. The purpose of adding preservatives or chelating agents in the formulation is to avoid any microbial contamination during storage.

For the purpose of present invention suitable iso-osmolality modifiers include but are not limited to carbohydrate, a polyhydric alcohol, or a combination thereof. Examples of iso-osmolality modifiers include sodium chloride, potassium chloride, sodium sulphate, glycerol, trehalose, mannitol, sorbitol, dextrose, lactose, and arginine

The iso-osmolality modifier for the purpose of present invention is a halogenide or mannitol.

The concentration of halogenide used in the present invention is from about 5 to 200 mM.

The osmolarity of the pharmaceutical compositions of the present invention is about 250-300 Osm/l.

Suitable chelating agents include but are not limited to one or more of edetic acid and its salts, or disodium dentate.

Examples of pH modifiers include but are not limited to one or more of acetate buffer, glutamate buffer, citrate buffer, prolamine buffers, carbonate buffers, phosphate buffer, hydrochloric acid, trolamine, sulfuric acid, sodium hydroxide solutions and any combination thereof.

The pH of the pharmaceutical compositions of the present invention is between 3-7.

The diluent for reconstitution may further comprise co-solvents, thickening agents, solubilizers, or antioxidants.

Suitable co-solvents include but are not limited to alcohol, glycerin, propylene glycol, and polyethylene glycol.

Suitable thickening agents or viscosity modifiers may comprise one or more of methylcellulose, carboxymethylcellulose, microcrystalline cellulose, ethylcellulose, hydroxyethylcellulose, hydroxypropylcellulose, hydroxypropylmethylcellulose, alginate, carageenan, xanthan gum, acacia, tragacanth, locust bean gum, guar gum, carboxypolymethylene, polyvinyl pyrrolidone, polyvinyl alcohol, poloxamer, magnesium aluminum silicate (veegum), bentonite, hectorite, povidone, maltitol, chitosan or combination thereof.

Suitable solubilizers are those known to ordinary skill in the art and include but not limited to one or more of glycofurol, polyethylene glycol, polyoxyethylene glycerol esters of fatty acids, such as Tagats; polooxylated castor oil, ethylene glycol esters, such as glycol stearate and distearate; propylene glycol esters, such as propylene glycol myristate; glyceryl esters of fatty acids, such as glyceryl stearates and monostearates; sorbitan esters, such as spans and tweens; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as Labrafils, Labrafacs, and Labrasols; cremophores; glycerol monocaprylate/caprate, such as Campmul CM 10; Gelucire, Capryol, Captex, Acconon, transcutol, triacetin, or TPGS (d-alpha tocopheryl polyethylene glycol succinate)

Suitable anti-oxidants include but are not limited to sodium metabisulfite, potassium metabisulfite, edentate, ascorbic acid and ascorbyl palmitate. Typically, the antioxidant may be present in the formulations in a concentration of from about 0.01% up to about 5% by weight of the total formulation.

The present invention is a solutions or suspension, emulsion, liposomes, microemulsion or gel.

The pharmaceutical composition of the present invention can be given in combination with non-opioid analgesics or other opioid analgesics. Non-opioid analgesics include but are not limited commonly used non-steroidal anti-inflammatory agents like ibuprofen, flurbiprofen, diclofenac, indomethacin, piroxicam, ketoprofen, etodolac, diflusinal, meloxicam, aceclofenac, fenoprofen, naproxen, celecoxib, rofecoxib or combination of foregoing. The other opioid analgesics but are not limited to codeine, meperidine, alfentanil, tapentadol, sufentanil, fentanyl, propoxyphene, levorphanol, hydromorphone, oxymorphone, hydrocodone, Oxycodone, methadone, naloxone, naltrexone or combination of foregoing.

The pharmaceutical compositions of the present invention are stable for at least three weeks at 25° C./60% RH.

The viscosity of the pharmaceutical composition of the present invention varies between 0.5-50 cps.

The pharmaceutical compositions of the present invention are suitable for multiple use, multiple times in one or different patients.

The pharmaceutical compositions of the present invention are administered to a patient in need of pain relief.

The pharmaceutical composition, according to present invention, used for preventing or treating pain comprising diamorphine or a pharmaceutically acceptable salt thereof is obtained by reconstituting 144 mg or 320 mg of diamorphine hydrochloride with 10 ml of a diluent for reconstitution.

The compositions according to present invention are useful for preventing or treating painful conditions caused by surgical procedures, orthopaedic procedures, migraine, sickle cell crisis, or burn dressing change.

The present invention provides a pharmaceutical composition comprising diamorphine or a pharmaceutically acceptable salt thereof, wherein the composition is adapted for intranasal delivery of diamorphine or a pharmaceutically acceptable salt thereof using a nasal spray.

The pharmaceutical composition according to present invention is liquid or lyophilized powder for reconstitution. The lyophilized powder for reconstitution is reconstituted using diluent for reconstitution, which may further comprise co-solvents, thickening agents, solubilizers, or antioxidants.

The pharmaceutical composition according to present invention is obtained by reconstituting 144 mg or 320 mg of diamorphine hydrochloride with 10 ml or 5 ml of a diluent for reconstitution.

The pharmaceutical composition according to present invention are useful in treatment of pain caused by surgical procedures, orthopaedic procedures, migraine, sickle cell crisis, or burn dressing change.

The present invention provides a method of assembling a device suitable for administration to multiple patients a multi-dose pharmaceutical composition of nasal analgesic in the form of a nasal spray without cross contamination, wherein the method comprises: (a) attaching the modified nasal pump to the bottle by hand so that it fits tightly into a bottle to prevent leakage; (b) shaking the assembly of step (a) for reconstitution of the lyophilized nasal analgesic; (c) priming the assembly of step (b) to release the intended quantity of the reconstituted nasal analgesic; (d) inserting the nasal tip into the patient no. 1's nostril and directing the spray to the sidewall of the nose of patient; (e) replacing the used nasal tip of step (d) with new nasal tip; (f) priming the assembly to release the intended quantity of the reconstituted nasal analgesic, and (g) inserting the nasal tip into the patient no. 2's nostril and directing the spray to the sidewall of the nose of patient.

The kit according to present invention comprises a bottle containing lyophilized nasal analgesic, a tube containing diluent for reconstitution, modified nasal pump, nasal tips and pack insert or a label, which provides directions for assembling the kit and the subsequent use.

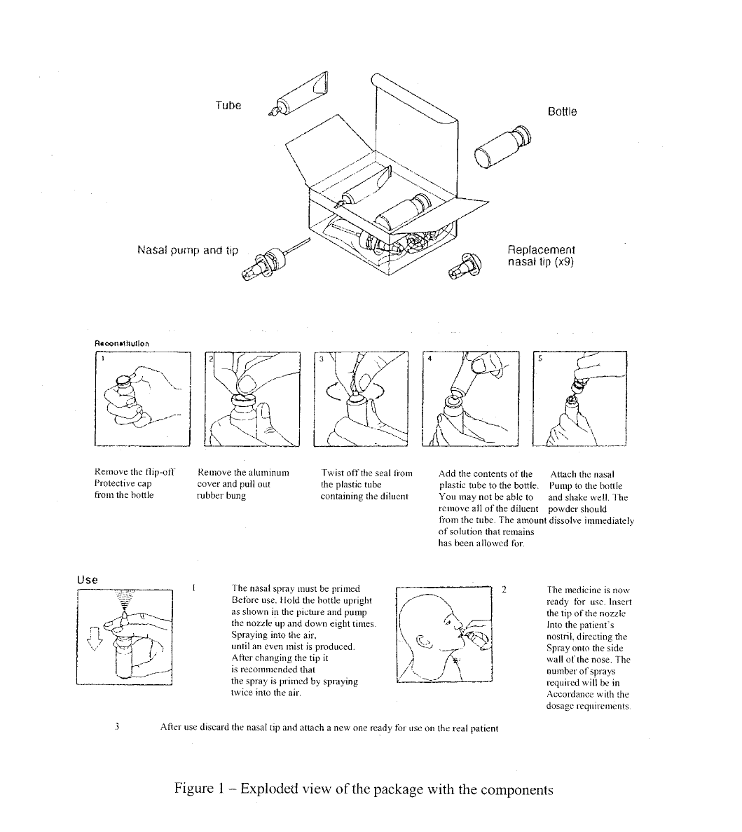

The kit of the present invention is in the form of a rectangular box, comprising 5 closed ends, four of which are on the sides and one in the bottom. The upper part of the rectangular box comprises three fold flaps, two of which arises from shorter edges of equal shape and size as to 10 meet at the center. Folding these two fold flaps ensures safe custody of the package contents. The flap arising from the longer edge of the upper portion of the rectangular box has a small protrusion that fits well into the groove when closed as to form a tight fit. (FIG. 1)

Upper portion of the kit has provision of two depressions that ensures tight fit of the tube and the bottle. The lower portion of the rectangular box has provisions for modified nasal pump and nasal tip and nine number of additional nasal tips for muti-dosing. (FIG. 1)

Depending on the type of lyophilized analgesic, intended dosage, and frequency of dosage, capacities of modified nasal pump, nasal tip and bottle may vary. The pump capacity chosen is such that it would deliver a certain predetermined quantity of the reconstituted liquid nasal analgesic. Some of the analgesics need to be administered in certain predetermined strengths and doses. For example, diamorphine dosage strength would be different for children in the range 12-30 kg and the other 30-50 kg. Typical dosage strengths 25 would be 0.1 mg/kg (±20%) over the weight range 12-50 kg.

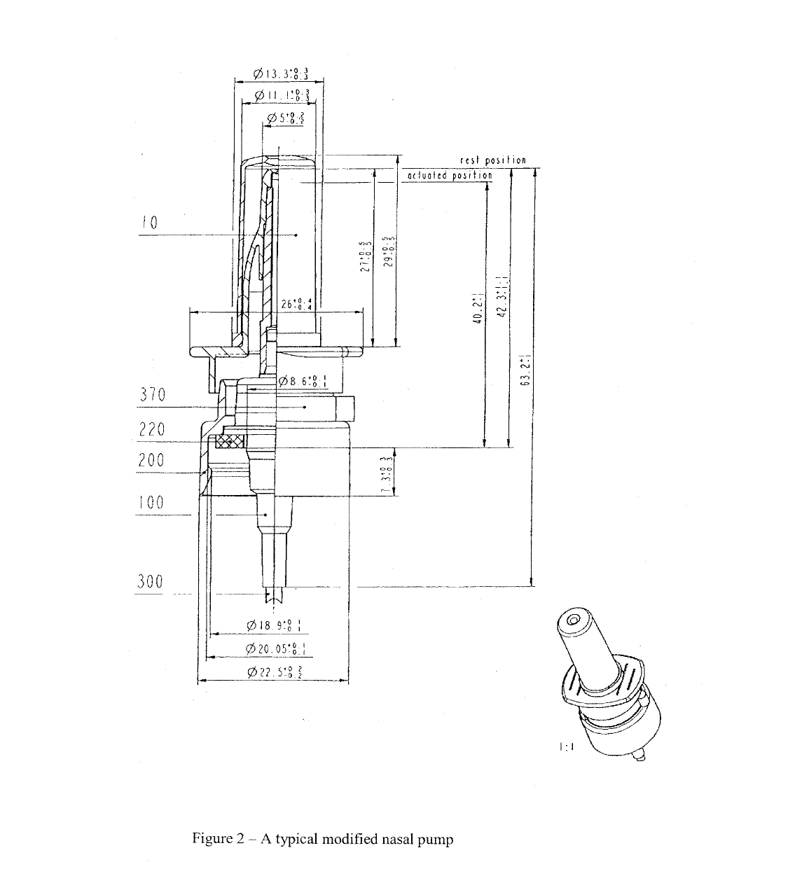

In another aspect of the present invention there is provided a device of FIG. 2 comprising

a) Nasal tip with cap
b) Modified nasal pump
c) Delivery tube

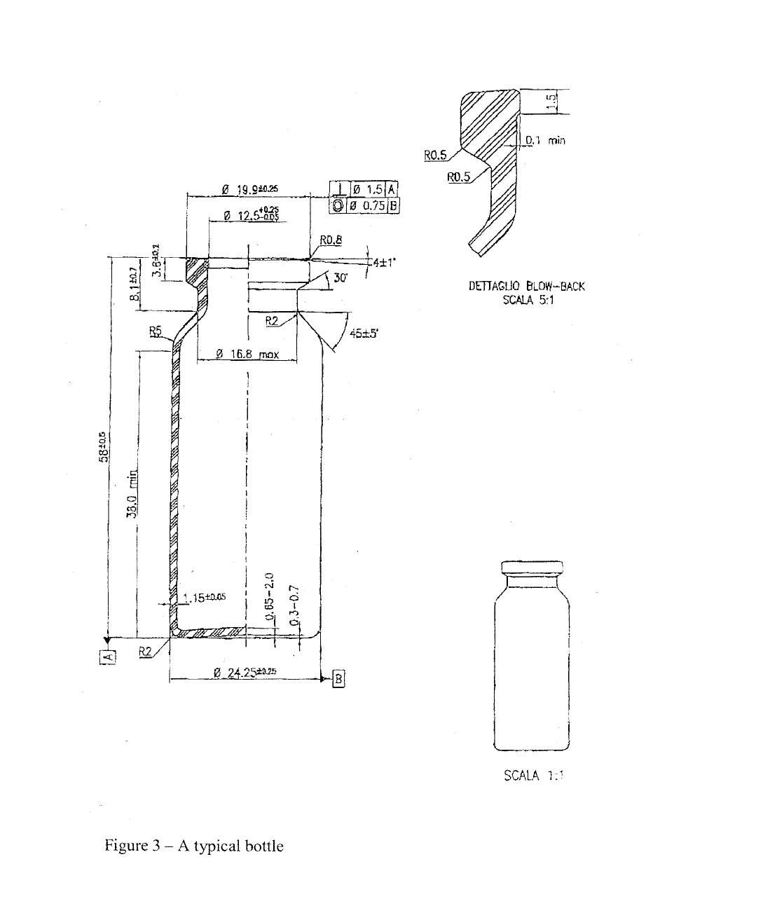

In an embodiment, a typical modified nasal pump (0.05 mL capacity) employed for attachment to 17 mL bottle is exhibited in FIG. 2. The various components of the pump are numbered in FIG. 2 such as nasal tip with cap (10), delivery tube (300) and the like. Suitable changes have been effected in the main attachment component (200) of the pump as to make a tight fit into the bottle exhibited in FIG. 3. To achieve this, allowable tolerances in variation in diameter of the fitting circular components requires to be as less as possible. It may be noticed that the outer diameter is 19.9 mm (±0.25 mm) at the entry of the bottle. The inner diameter of the component of the modified nasal pump that fits tightly onto the mouth of the bottle by hand is 20.05 mm (±0.1 mm)

The changes effected in the attachment component (200) are such that it is devoid of screw threading or crimping on by machine. The changes effected in the component of the pump are essential as normally threads would be present in a bottle. Further, the changes effected on the pump attachment calls for modification of the existing mould.

The detailed method of assembly and use of nasal spray shown in FIG. 1 comprises:
1. removal of “flip off tear off” protective cap from the bottle,
2. removal of the aluminum cover and pulling out the rubber bung,
3. twisting off the seal from the tube containing the diluent for reconstitution,
4. adding the contents of the tube to the bottle. It may not be possible to remove all of the diluent from the tube.

In yet another aspect of the invention there is provided a method of assembling the kit (FIG. 1) so as to form a device suitable for administration of multi-dose pharmaceutical composition of nasal analgesic in the form of a nasal spray, wherein the method comprises
a) attaching the modified nasal pump to the bottle by hand so that it fits tightly into a bottle to prevent leakage,
b) shaking the assembly of step a) for reconstitution of the lyophilized nasal analgesic,
c) priming the assembly of step b) to release the intended quantity of the reconstituted nasal analgesic,
d) inserting the nasal tip into the patient's nostril and directing the spray to the sidewall of the nose.

Before the usage of the reconstituted liquid, priming needs to be carried out for adequate number of times. Replacement of nasal tip before use on a new patient is an additional requirement for its effective administration. For example, a typical analgesic such as reconstituted diamorphine in 0.05 mL capacity modified nasal pump and 17 mL bottle, the components assemblage, require priming eight times to ensure that the pump is fully primed and releases 50 ul, each time it is used. Normal use by a patient is about 2-4 sprays. Twice priming after new nasal tip replacement is an additional feature. Almost 10 treatments per device can be repeated by employing 0.05 rnL modified nasal pump and 17 mL bottle.

In yet another aspect of the invention there is provided a method of assembling the kit (FIG. 1) so as to form a device suitable for administration to multiple patients a multi-dose pharmaceutical composition of nasal analgesic in the form of a nasal spray, wherein the method comprises
a) attaching the modified nasal pump to the bottle by hand so that it fits tightly into a bottle to prevent leakage,
b) shaking the assembly of step a) for reconstitution of the lyophilized nasal analgesic,
c) priming the assembly of step b) to release the intended quantity of the reconstituted nasal analgesic,
d) inserting the nasal tip into the patient no. 1's nostril and directing the spray to the sidewall of the nose of patient,
e) replacing the used nasal tip of step d) with new nasal tip,
f) priming the assembly to release the intended quantity of the reconstituted nasal analgesic,
g) inserting the nasal tip into the patient no. 2's nostril and directing the spray to the sidewall of the nose of patient.

The step e) to g) can be repeated no. of times, each time replacing the used nasal tip with the new nasal tip for each new patient. This present invention not only provides a convenient method for administration to a health professional for administering the compositions to multiple patients but also reduces the risk of cross-contamination of a formulation from multiple patient administrations by using replacable tips after every patient administration. The pharmaceutical compositions of the present invention were found to be effective in pediatric population.

The present invention is further illustrated by the following examples which are provided merely to be exemplary of the invention and do not limit the scope of the invention. Certain modifications and equivalents will be apparent to those skilled in the art and are intended to be included within the scope of the present invention.

### EXAMPLES

### Example 1

TABLE 1
Nasal Composition

| S.N. | Ingredients | Quantity (% w/v) |
|---|---|---|
| 1. | Lyophilized Diamorphine | 0.5-50 |
| | Hydrochloride | |
| 2. | Sodium Chloride | 0.2-10 |
| 3. | Hydrochloric acid | pH 5.0 ± 0.2 |
| 4. | Sodium Hydroxide | pH 5.0 ± 0.2 |
| 5. | Purified Water | 100 |

### Example 2

TABLE 2
Nasal Composition

| S.N. | Ingredients | Quantity (% w/v) |
|---|---|---|
| 1. | Lyophilized Diamorphine | 0.5-50 |
| | Hydrochloride | |
| 2. | Diluent For Reconsitution | q.s. 10 ml |
| 3. | Mannitol | 0.2-20 |
| 4. | Hydrochloric acid | pH 5.0 ± 0.2 |
| 5. | Sodium Hydroxide | pH 5.0 ± 0.2 |
| 6. | Purified Water | 100 |

TABLE 3
Composition for diluent for reconstitution

| S.N. | Ingredients | Quantity |
|---|---|---|
| 1. | Benzalkonium chloride (50% w/v) | 0.2-6 ml |
| 2. | Disodium edetate | 0.2-20 mg |
| 3. | Mannitol | 0.2-20 mg/ml |
| 4. | Hydrochloric acid | pH 5.0 ± 0.2 |
| 5. | Sodium Hydroxide | pH 5.0 ± 0.2 |
| 6. | Purified Water | 10 ml |

### Example 3

### Stability Studies

The reconstituted diamorphine solution of Example 2 as per table 2 and 3 was prepared and alongwith the attached nasal spray device was subjected to stability studies at 25° C./60% RH and at 2-8° C. The samples were assayed initially, their after a week intervals after preparation. The results are disclosed in Table 3. During stability the compatibility of all the exposed components of the nasal spray device with the diamorphine solution was also checked.

Please insert the results of the stability tests.

It was observed that there is not a substantial decrease in the assay over three-week period. The observed decrease in assay is consistent with the deacetylation of diamorphine, which occurs in aqueous solution.

### Example 4

### Clinical Study: An Open Label Single Dose Pharmacokinetic Study of Diamorphine Hydrochloride Nasal Spray in Children

An open label, single dose, sparse sampling pharmacokinetic dentate study in children aged from 1-<16 years attending hospital clinics for minor day surgery or laser treatment and requiring intravenous cannulation and opiate analgesia for their routine clinical management. The cannulation site on the child's arm was numbed with local anaesthetic cream, and a cannula was inserted. The child was then anaesthetized and a baseline blood sample taken. The child received 1-3 sprays of intranasal diamorphine, depending on their weight, and another blood sample was taken as soon as possible (from 0-10 minutes) post dose. Up to four further pharmacokinetic blood samples were taken from the cannula for the purposes of the study spaced out over the following time windows: 10-20 minutes, 20-30 minutes, 30-45 minutes, 45-60 minutes, 60-120 minutes and between 120 minutes post dose and removal of the cannula. Plasma acetylmorphine, morphine, morphine-3-glucuronide, and morphine-6-glucuronide concentrations were determined. The data from all children taking part in the trial was pooled and analysed together to avoid excessive blood sampling. Population pharmacokinetic parameters were estimated by using a non-linear mixed effects dentate approach. Adverse events, vital signs, nasal tolerance and pain scores were also monitored.

Male or female aged from 3 months-<16 years of age, attending the hospital for routine elective surgery, and in the clinical opinion of the clinician in charge, required opiate analgesia for pain relief were selected for conducting study. The study was conducted in 57 subjects. Out of 57 subjects, 56 subjects completed the study (53 aged 1-11 years, 3 aged 12-<16 years).

Diamorphine Hydrochloride Nasal Spray comprising diamorphine hydrochloride (144 mg or 320 mg), freeze dried powder reconstituted with diluent for reconstitution (10 ml) and administered in an intranasal spray device as a single dose of 0.06 mg/kg to 0.1 mg/kg.

### Results:

For assessing the effectiveness of the Diamorphine Hydrochloride Nasal Spray pain scores were measured. The investigators or nurses measured the FLACC scores. The total score was based on 5 categories: (F) Face; (L) Legs; (A) Activity; (C) Cry; (C) Consolability; are scored between 0-1 giving a total pain score between 0 and 5.

Assessments were made for up to 3.5 hours (3 hours after surgery). The majority of assessments were made at earlier time points (0 mins (38 children), 30 min (32 children) and 1 hour (22 children), after this time the number of assessments was reduced. The results are reproduced in Table 5.

Pain scores were generally low, with a mean (SD) FLACC score of 0.4 (0.94) at 0 minutes (1 hr post-surgery). The maximum pain score reported at this time point was 4, and minimum 0. At 30 min (1.5 hr post-surgery) the mean (SD) pain score was also low (0.6 [1.76]), although the maximum score seen was 8 (minimum 0). Pain was reported at the 1.5 hr (2.5 hr post-surgery) time point, mean (SD) 0.1 (0.64). After 1.5 hr pain was not reported (all pain scores rated as 0). In addition the number of children in whom assessments were made reduced over time (13 children at 1.5 hr, 7 children at 2 hr, 3 children at 2.5 hr, 1 child at 3 hr, 1 child at 3.5 hr).

TABLE 5
FLACC Total Pain Scores (Safety Population)

| | | Diamorphine Hydrochloride Nasal Spray | | |
|---|---|---|---|---|
| | | Age Group | Age Group | |
| Actual time | | 1-11 years | 12-<16 years | Overall |
| [1] | Statistics | (N = 53) | (N = 3) | (N = 56) |
| 0 min | n | 36 | 2 | 38 |
| | Mean | 0.4 | 0.0 | 0.4 |
| | SD | 0.96 | 0.00 | 0.94 |
| | Minimum | 0 | 0 | 0 |
| | Median | 0.0 | 0.0 | 0.0 |
| | Maximum | 4 | 0 | 4 |
| 30 min | n | 30 | 2 | 32 |
| | Mean | 0.4 | 2.5 | 0.6 |
| | SD | 1.61 | 3.54 | 1.76 |
| | Minimum | 0 | 0 | 0 |
| | Median | 0.0 | 2.5 | 0.0 |
| | Maximum | 8 | 5 | 8 |
| 1 hr | n | 21 | 1 | 22 |
| | Mean | 0.1 | 0.0 | 0.1 |
| | SD | 0.65 | | 0.64 |
| | Minimum | 0 | 0 | 0 |
| | Median | 0.0 | 0.0 | 0.0 |
| | Maximum | 3 | 0 | 3 |
| 1 hr 30 | n | 13 | 0 | 13 |
| | Mean | 0.0 | | 0.0 |
| | SD | 0.00 | | 0.00 |
| | Minimum | 0 | | 0 |
| | Median | 0.0 | | 0.0 |
| | Maximum | 0 | | 0 |
| 2 hr | n | 6 | 1 | 7 |
| | Mean | 0.0 | 0.0 | 0.0 |
| | SD | 0.00 | | 0.00 |
| | Minimum | 0 | 0 | 0 |
| | Median | 0.0 | 0.0 | 0.0 |
| | Maximum | 0 | 0 | 0 |
| 2 hr 30 | n | 3 | 0 | 3 |
| | Mean | 0.0 | | 0.0 |
| | SD | 0.00 | | 0.00 |
| | Minimum | 0 | | 0 |
| | Median | 0.0 | | 0.0 |
| | Maximum | 0 | | 0 |
| 3 hr | n | 1 | 0 | 1 |
| | Mean | 0.0 | | 0.0 |
| | SD | | | |
| | Minimum | 0 | | 0 |
| | Median | 0.0 | | 0.0 |
| | Maximum | 0 | | 0 |
| 3 hr 30 | n | 1 | 0 | 1 |
| | Mean | 0.0 | | 0.0 |
| | SD | | | |
| | Minimum | 0 | | 0 |
| | Median | 0.0 | | 0.0 |
| | Maximum | 0 | | 0 |

[1] Actual time window (+/−15 minutes) following one hour after operation.
N = the number of patients in the population.
N = the number of patients with a measurement.

### Adverse Events:

The incidence of adverse events was very low and unremarkable. There were 42 adverse events reported by 23 patients. 41% of all patients reported at least one event; 40% of the younger children (1-11 years), and 67% of the older children (12-<16). None of these events led to withdrawal or death of a patient. The most commonly reported body system for adverse events was gastrointestinal disorders with 6 patients reporting 16 events, in all cases post-operatively (vomiting [6 patients; definite (1), probably (2) and possibly (3) related to the IMP]), nausea [3 patients; possibly related (3)] and haematemesis [1 patient; possibly related to the IMP]). All were considered mild except the haematemesis (severity moderate) (the child had most likely swallowed blood during the dental procedure). Table 6 summarizes the number of events reported.

TABLE 6
Adverse Event Overview by Number of Events (Safety Population)

| | | | | Age Group 12-<16 years (N = 3) | | | |
|---|---|---|---|---|---|---|---|
| | Age Group 1-11 years (N = 53) | | | (TOTAL [Patient Numbers]) | | | |
| | (TOTAL [Patient Numbers]) | | | | | | Total number of events |
| Body System | Mild | Moderate | Severe | Mild | Moderate | Severe | (number of patients) |
| Gastrointestinal | 15 | 1 | 0 | 0 | 0 | 0 | 16 (6) |
| disorders | 1/001, ½, | 2/010 | | | | | |
| | 1/009, | | | | | | |
| | 2/009, | | | | | | |
| | 2/009 | | | | | | |
| | 2/010, | | | | | | |
| | 2/011, | | | | | | |
| | 2/011, | | | | | | |
| | 2/011, | | | | | | |
| | 2/011, | | | | | | |
| | 2/011, | | | | | | |
| | 2/011, | | | | | | |
| | 2/012, | | | | | | |
| | 2/012, | | | | | | |
| | 2/012 | | | | | | |
| Injury, | 10 | 1 | 2 | 0 | 1 | 0 | 14 (14) |
| poisoning | 1/012, | 2/001 | 1/001 | | 2/002 | | |
| and procedural | 1/022, | | 1/010 | | | | |
| complications | 1/024, | | | | | | |
| | 1/031^{#}, | | | | | | |
| | 1/034 | | | | | | |
| | 1/044, | | | | | | |
| | 1/045, | | | | | | |
| | 2/006, | | | | | | |
| | 2/009, | | | | | | |
| | 2/012 | | | | | | |
| Respiratory, | 9 | 0 | 0 | 1 | 0 | 0 | 10 (9) |
| thoracic and | ¼, 1/038, | | | 1/04 | | | |
| mediastinal | 1/040, | | | 1 | | | |
| disorders | 2/004, | | | | | | |
| | 2/007 | | | | | | |
| | 2/009, | | | | | | |
| | 2/010, | | | | | | |
| | 2/010, | | | | | | |
| | 2/011 | | | | | | |
| Nervous | 2 | 0 | 0 | 0 | 0 | 0 | 2 (1) |
| system | 2/011, | | | | | | |
| disorders | 2/011 | | | | | | |
| Total number | 36 (19) | 2 (2) | 2 (2) | 1 (1) | 1 (1) | 0 (0) | 42 Total |
| of events | | | | | | | |
| (number of | | | | | | | |
| patients) | | | | | | | |
| | | | | | | | 16 Non-related [1] |
| | | | | | | | 26 Related [2] |
| | | | | | | | 37 Mild |
| | | | | | | | 3 Moderate |
| | | | | | | | 2 Severe |

N = the number of patients in the population.
^{#}3 patients completed the study twice (patient 1/016 as 1/023, patient 1/013 as 1/026 and patient 1/003 as 1/031).
[1] Non-related events are defined as Unlikely or Unrelated to study medication.
[2] Related events are defined as Definite, Probably or Possibly related to study medication.

### Respiratory Depression:

There was no respiratory depression in terms of clinically notable changes in vital signs was observed during the study.

The single dose of 0.1 mg/kg body weight of diamorphine formulated into the pharmaceutical composition when administered intranasally to a patient in need of pain relief was found to be safe, effective and free from the major side effects such as respiratory depression when compared to 0.1 mg/kg of diamorphine lyophillisate for injection.

1. A method of reducing the respiratory depressant effect of diamorphine wherein the method comprises administering intranasally a single dose pharmaceutical composition comprising (a) lyophilized diamorphine or pharmaceutically acceptable salts thereof, and (b) a diluent for reconstitution; wherein the diluent for reconstitution comprises a preservative, a chelating agent, one or more osmolarity modifiers to maintain the osmolarlity of the said composition between 250-500 Osm/L and pH modifiers to maintain the pH of the said composition between 3.5 to 6.5, wherein the dose of diamorphine is 0.1 mg/kg body weight.

2. The method of claim 1, wherein diamorphine or pharmaceutically acceptable salts thereof is diamorphine hydrochloride.

3. The method of claim 1, wherein one or more osmolarity modifiers are selected from the group consisting of a halogenide, carbohydrate, a polyhydric alcohol or combination of foregoing.

4. The method of claim 3, wherein one or more osmolarity modifiers are selected from the group consisting of sodium chloride, potassium chloride, glycerol, trehalose, mannitol, sorbitol, dextrose, lactose, and arginine.

5. The method of claim 3, wherein one or more osmolarity modifier is a halogenide in concentration range of 5 to 200 mM.

6. The method of claim 1, wherein one or more preservatives are selected from the group consisting of m-cresol, phenol, alcohol, benzyl alcohol, methyl-, ethyl-, propyl- and butyl-paraben, thiomersal, chlorobutanol, benzalkonium or any combination thereof.

7. The method of claim 1, wherein chelating agent is selected from the group consisting of edetic acid and its salts, or disodium dentate.

8. The method of claim 1, wherein pH modifiers are selected from the group consisting of acetate buffer, glutamate buffer, citrate buffer, prolamine buffers, carbonate buffers, phosphate buffer, sodium hydroxide, hydrochloric acid and any combination thereof.

9. The method of claim 1, wherein the pharmaceutical composition is administered to a patient in need of pain relief.

10. A method for treating pain, said method comprising administering intranasally a pharmaceutical composition comprising diamorphine or a pharmaceutically acceptable salt thereof, reconstituted with a diluent for reconstitution, said composition adapted for a delivery of a single dose of diamorphine or a pharmaceutically acceptable salt thereof in an amount of 0.1 mg diamorphine or a pharmaceutically acceptable salt thereof per kg body weight of a patient.

11. The method according to claim 10, wherein the composition is obtained by reconstituting 144 mg or 320 mg of diamorphine hydrochloride with 10 ml or 5 ml of a diluent for reconstitution.

12. The method according to claim 10, wherein the pain is caused by surgical procedures, orthopaedic procedures, migraine, sickle cell crisis, or burn dressing change.

13. A pharmaceutical composition comprising diamorphine or a pharmaceutically acceptable salt thereof, wherein the composition is adapted for intranasal delivery of diamorphine or a pharmaceutically acceptable salt thereof using a nasal spray capable of delivering diamorphine or a pharmaceutically acceptable salt thereof in an amount of 0.1 mg/kg body weight.

14. A method of assembling a device suitable for administration to multiple patients a multi-dose pharmaceutical composition of nasal analgesic in the form of a nasal spray, wherein the method comprises: (a) attaching the modified nasal pump to the bottle by hand so that it fits tightly into a bottle to prevent leakage; (b) shaking the assembly of step (a) for reconstitution of the lyophilized nasal analgesic; (c) priming the assembly of step (b) to release the intended quantity of the reconstituted nasal analgesic; (d) inserting the nasal tip into the patient no. 1's nostril and directing the spray to the sidewall of the nose of patient; (e) replacing the used nasal tip of step (d) with new nasal tip; (f) priming the assembly to release the intended quantity of the reconstituted nasal analgesic, and (g) inserting the nasal tip into the patient no. 2's nostril and directing the spray to the sidewall of the nose of patient.

15. A kit for preparation of a multi-dose pharmaceutical composition of nasal analgesic in the form of a nasal spray comprising a bottle containing lyophilized nasal analgesic, a tube containing diluent for reconstitution, modified nasal pump, one or more disposable nasal tips, and a pack insert or a label providing directions for assemble the kit and its use.

16. A method for reducing the cross-contamination while using a multi-dose pharmaceutical composition of nasal analgesic in the form of a nasal spray in multiple patients by providing a kit comprising multi-dose pharmaceutical composition and replaceable nasal tips; wherein the method comprises (a) inserting the nasal tip of the nasal spray comprising the pharmaceutical composition into the patient no. 1's nostril and directing the spray to the sidewall of the nose of patient; (b) replacing the used nasal tip of step (a) with new nasal tip; (c) inserting the nasal tip into the patient no. 2's nostril.
